(19) United States
(12) Patent Application Publication
(10) Pub. No.: US 2023/0215574 A1
(43) Pub. Date: Jul. 6, 2023

(54) DETERMINING AN AIRBORNE AND/OR AEROSOL PATHOGEN RISK EXPOSURE

(71) Applicant: SAINT-GOBAIN ECOPHON AB, Hyllinge (SE)

(72) Inventors: Ola KARLSSON, Lund (SE); Erling NILSSON, Lund (SE); Pierre CHIGOT, Helsingborg (SE); Francois MICHEL, Sceaux (FR)

(73) Assignee: SAINT-GOBAIN ECOPHON AB, Hyllinge (SE)

(21) Appl. No.: 17/999,900

(22) PCT Filed: May 21, 2021
(86) PCT No.: PCT/EP2021/063576

§ 371 Date: Nov. 25, 2022

(30) Foreign Application Priority Data
Jun. 5, 2020 (SE) 2050656-4

(51) Int. Cl. G16H 50/30; G16H 50/80; H04W 4/021

(52) U.S. Cl. CPC G16H 50/30; G16H 50/80; H04W 4/021

(57) ABSTRACT

A computer implemented method for determining an airborne and/or aerosol pathogen risk exposure includes determining an acoustical parameter for a portion of a building, recording, using a microphone in a mobile electronic device located in the portion of the building, sound pressure level over time originating from speech in the portion of the building, and determining the airborne and/or aerosol pathogens risk exposure as a function of the recorded sound pressure over time and the determined acoustical parameter.

### TECHNICAL FIELD

The present invention relates to determining an airborne and/or aerosol pathogen risk exposure.

### BACKGROUND

Particles expelled during human expiratory events, such as sneezing, coughing, talking, and breathing, serve as vehicles for respiratory pathogen transmission. In particular, small particles are believed to be generated during breathing and talking from the mucosal layers coating the respiratory tract. Despite their size, small particles are larger enough to carry a large variety of respiratory pathogens.

It has been recognized that small particles can be more infectious than larger droplets. They indeed persist in the air for longer time periods before setting by gravity, thus increasing the probability of inhalation by susceptible individuals. They have a larger probability of penetrating further into the respiratory tract. They can be released dramatically in larger quantity during speech compared to coughing.

According to some studies, the rate of particle emission during normal human speech seems to correlate with the loudness (amplitude) of vocalization, for low to high amplitudes, regardless of the language spoken.

When an epidemic is spreading, the fact that contagious particles may be emitted in a conversational environment is often overlooked as many people focus mainly on coughing and sneezing as main potential sources of contamination. Furthermore, for most disease, a person is contagious well before she has symptoms, and, today, there is no solution to track the potentially contagious conversational environments that a person may have encounter before.

Hence, there is a need to be able to evaluate the risk of a conversational environment to a communication vector for fluids emitted from human airways, and may help to track the history of the potential contagious conversational environment a person have encountered during its day-to-day activities.

### SUMMARY OF THE INVENTION

It is an object to mitigate, alleviate or eliminate one or more of the above-identified deficiencies in the art and disadvantages singly or in any combination and solve at least the above-mentioned problem.

According to a first aspect a computer implemented method for determining an airborne and/or aerosol pathogen risk exposure is provided. The method comprising: determining an acoustical parameter for a portion of a building; recording, by means of a microphone in a mobile electronic device located in the portion of the building, sound pressure level over time originating from speech in the portion of the building; determining the airborne and/or aerosol pathogens risk exposure as a function of the recorded sound pressure over time and the determined acoustical parameter.

Using the computer implemented method an estimate of the probability of a conversational environment, i.e. an environment of speaking people, to be a communication vector for fluids emitted from human airways may be made. The present invention relies on the processing of a record of the sound pressure level of a conversational environment a person is exposed to. The sound pressure level is typically determined over a range of frequencies which are characteristics of the human voice. This in order to estimate a probability level of potential contagiousness by comparing the recorded sound pressure level with an acoustical parameter of the portion of a building wherein the conversational environment is formed. Sound pressure level is a local pressure deviation from the ambient atmospheric pressure. This deviation is caused by a sound wave that may be emitted by a human, an animal or an object. It can be measure with a microphone. Many signal processing methods are available in the art for determining sound pressure level. Further, signal processing available in the art may also be used to distinguish between different kind of emitters. Hence, sound pressure level originating from human speech may be filtered from other sound. The method may be highly valuable during the spreading of an epidemic as it allows to evaluate the level of contagiousness of a conversational environment of an exposed subject. The method may be used to warn a person of the risk of contamination.

The act of determining the acoustical parameter for the portion of the building may be performed using the microphone of the mobile electronic device located in the portion of the building.

The act of determining an acoustical parameter for the portion of the building may comprise determining more than one acoustical parameter.

The method may further comprise determining a muffled or unmuffled condition of the microphone. Upon determining a muffled condition, the act of determining the airborne and/or aerosol pathogens risk exposure may comprise compensating for the muffled condition.

Presence of a mobile electronic device in the portion of the building may be determined using a wireless communication-based positioning function.

The method may further comprise logging time resolved presence information for persons being present in the portion of the building by identifying presence of mobile electronic devices, associated with the respective person, in the portion of the building. Hence, based on a geographical position tracking system, persons exposure to potentially contagious environments may be tracked. This may be valuable information to fight an epidemic by reconstructing the contamination history, and/or to check whether health regulations, e.g. social distancing, have been complied with.

The method may further comprise registering whether a person being present in the portion of the building was infected by a disease while being in the portion of the building. Upon a person being present in the portion of the building was infected by a disease, performing the act of determining the airborne and/or aerosol pathogens risk exposure.

The act of recording sound pressure level over time originating from speech in the portion of the building may comprise recording individual sound pressure levels over time originating from speech from different persons being present in the portion of the building. Accordingly, sound pressure level originating from individual persons may also be distinguish from each other using signal processing available in the art. Hence, the recorded sound pressure level may be processed so that to identify different voices and sort the related speaking people according to their likeliness to emit particles, e.g. respiratory droplets, that may serve as vehicles for respiratory pathogen transmission.

The act of recording sound pressure level over time is performed by means of a plurality of microphones in a plurality of mobile electronic devices located in the portion of the building.

The mobile electronic device may comprise one or more of: a mobile phone, a smart watch, a laptop, and a tablet.

According to a second aspect a non-transitory computer readable recording medium is provided. The non-transitory computer readable recording medium comprising program code portions recorded thereon which when executed on a device having processing capability is configured to perform the method of the first aspect.

The above-mentioned features of the method of the first aspect, when applicable, apply to this second aspect as well. In order to avoid undue repetition, reference is made to the above.

According to a third aspect an electronic system configured to determining an airborne and/or aerosol pathogen risk exposure is provided. The electronic system comprising: a microphone; and circuitry. The circuitry is configured to execute: an acoustical parameter determining function configured to determine an acoustical parameter for a portion of a building; a sound recording function configured to record, by means of the microphone, sound pressure level over time originating from speech in the portion of the building; and a risk evaluation function configured to determine the airborne and/or aerosol pathogens risk exposure as a function of the recorded sound pressure over time and the determined acoustical parameter.

The microphone may be comprised in a mobile electronic device located in the portion of the building.

The circuitry may be comprised in the mobile electronic device. Hence, the electronic system may be implemented as mobile electronic device.

The circuitry may be comprised in a server being in wireless communication with the mobile electronic device.

The circuitry may be in part comprised in the mobile electronic device and in part comprised in a server being in wireless communication with the mobile electronic device.

The above-mentioned features of the method of the first aspect, when applicable, apply to this third aspect as well. In order to avoid undue repetition, reference is made to the above.

A further scope of applicability of the present invention will become apparent from the detailed description given below. However, it should be understood that the detailed description and specific examples, while indicating preferred embodiments of the invention, are given by way of illustration only, since various changes and modifications within the scope of the invention will become apparent to those skilled in the art from this detailed description.

Hence, it is to be understood that this invention is not limited to the particular component parts of the device described or acts of the methods described as such device and method may vary. It is also to be understood that the terminology used herein is for purpose of describing particular embodiments only, and is not intended to be limiting. It must be noted that, as used in the specification and the appended claim, the articles “a,” “an,” “the,” and “said” are intended to mean that there are one or more of the elements unless the context clearly dictates otherwise. Thus, for example, reference to “a unit” or “the unit” may include several devices, and the like. Furthermore, the words “comprising”, “including”, “containing” and similar wordings does not exclude other elements or steps.

### BRIEF DESCRIPTION OF THE DRAWINGS

The above and other aspects of the present invention will now be described in more detail, with reference to appended figures. The figures should not be considered limiting; instead they are used for explaining and understanding.

As illustrated in the figures, the sizes of layers and regions may be exaggerated for illustrative purposes and, thus, are provided to illustrate the general structures. Like reference numerals refer to like elements throughout.

FIG. 1 illustrates a portion of a building wherein a plurality of persons are present, each person being associated with a mobile electronic device.

FIG. 2 is a block scheme of a computer implemented method for determining an airborne and/or aerosol pathogen risk exposure.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and to fully convey the scope of the invention to the skilled person.

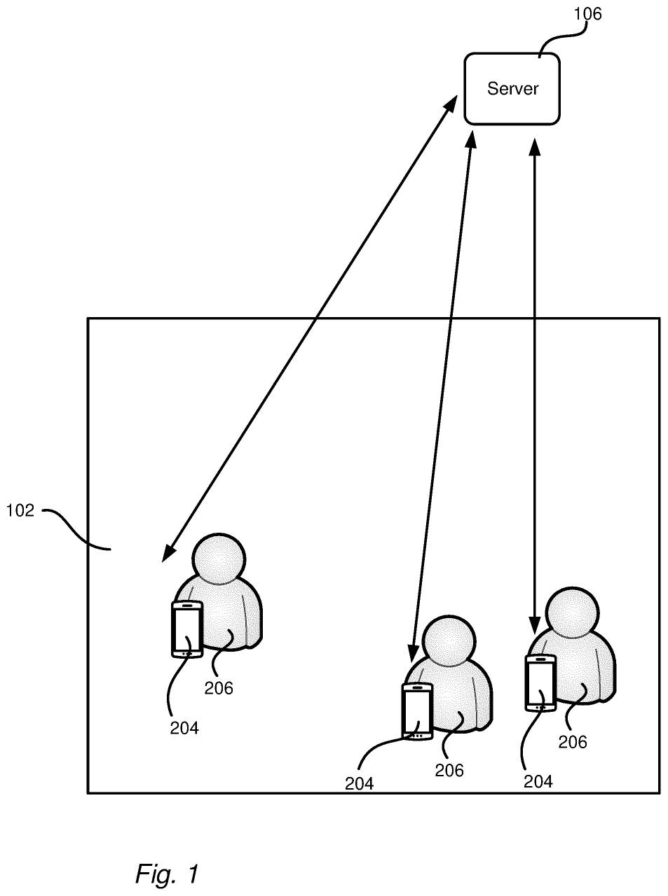

FIG. 1 illustrates a portion 102 of a building. The portion 102 of the building may be a room of the building 100, wherein a room is a space of the building delimited by a floor, a ceiling and walls. However, a portion 102 of the building may be part of a large room of the building. Hence, a large room of the building 100 may be divided into a plurality of portions 102.

A plurality of persons 206 may be present in the portion 102 of the building. The persons 206 may speak with each other. In case one or more of the persons 206 is infected with a disease that may be spread via respiratory pathogens there might be an airborne and/or aerosol pathogen risk of exposure for the other persons present in the portion 102 of the building. It has been found that airborne and/or aerosol pathogen risk exposure increases with increased level of speech. A person speaking relatively loudly will expel aerosols possible comprising pathogens further than a person speaking relatively gently.

Today, persons typically carry around one or more mobile electronic devices 204. Such mobile electronic devices 204 are typically personal, i.e. belonging to a specific person. Hence, a specific mobile electronic device 204 may be associated with a specific person. Data linking a specific mobile electronic device 204 to a specific person may be stored in the specific mobile electronic device 204 or elsewhere, e.g. in a database remote from the mobile electronic device 204. The mobile electronic device 204 may e.g. be a smartphone, a smartwatch, a laptop, or a tablet. The mobile electronic device 204 comprises a microphone. The microphone may be used to register and/or record sound in the vicinity of the mobile electronic device 204. Further, there are numerous methods available in the art for determining whether a mobile electronic device 204 is located in a portion of a building. Typically, such a method is based on a wireless communication-based positioning function. Any such method may be used in order to determine location of mobile electronic devices 204 in the portion 102 of the building.

The mobile electronic devices 204 may be used to log which persons that are present in the portion 102 of the building. Hence, time resolved presence information for persons being present in the portion 102 of the building may be logged. Such logging may take place in a server 106. Logging of such time resolved presence information for persons being present in the portion 102 of the building may later on be used in infection tracing.

In line with the present invention the mobile electronic devices 204 may also be used to determine an airborne and/or aerosol pathogen risk exposure. As mentioned above it has been found that airborne and/or aerosol pathogen risk exposure increases with increased level of speech. Accordingly, there is a correlation between a sound pressure level due to speech and an amount of contaminating emissions from a speaker and an increased dose/risk for the other persons in the portion 102 of the building. The higher the sound pressure level, the higher the exposure and the dose for the others. A sound pressure level, i.e. a conversational environment, in the portion 102 of the building may be recorded using one or more of the microphones in the mobile electronic devices 204 being present in the portion 102 of the building. This by recording, by means of a microphone in a mobile electronic device 204 located in the portion 102 of the building, sound pressure level over time in the portion 120 of the building. The recorded sound pressure level over time may be stored locally on a memory, e.g. a digital storage medium, of the mobile electronic device 204 being used to record the sound pressure level over time. Alternatively, or in combination, the recorded sound pressure level over time may be stored remotely on a memory, e.g. a digital storage medium, of the server 106. Preferably, the sound pressure level is filtered so that sound pressure level originating from speech is recorded. The filtering may e.g. be made using a voice discrimination algorithm. The voice discrimination algorithm separate voice sources from other noise in the portion 102 of the building. The filtering may be made directly at the mobile electronic device 204 and/or at the server 106.

However, a recoded sound pressure level over time in the portion 120 of the building, even after filtering out sound originating from speech, is typically not enough to determine airborne and/or aerosol pathogen risk exposure. In indoor environment, the sound pressure level of human voice during speech may depend on various factors, such as the size of the indoor, the amount of sound absorption in the room, and/or the speech behavior of speaker. Hence, properties of the portion 102 of the building will influence the sound pressure level recorded by the microphone. Especially, acoustical properties of the portion 102 of the building. Hence, in order to determining the airborne and/or aerosol pathogens risk exposure also an acoustical parameter for the portion 102 of a building may be needed. The acoustical parameter may be a parameter related to the acoustical damping present in the portion 102 of the building. One such acoustical parameter may e.g. be the sound strength parameter, G. The sound strength parameter, G, is a room acoustical parameter used to investigate the sound distribution in a portion of a building. The G parameter may e.g. be used to compare the loudness between different portions of a building. ISO 3382-1 describes several methods to measure G. The acoustical parameter, e.g. G, may be determined using a sound recording performed by means of one or more microphones of one or more mobile electronic devices present in the portion of the building. The acoustical parameter, e.g. G, may be determined by other known methods, see e.g. ISO 3382-1. When the acoustical parameter for a specific portion of a building is determined it may be stored in the server 106, particularly together with an identifier for the specific portion of the building. The stored acoustical parameter for a specific portion of a building may be dynamically updated in connection with new measurement(s) of the acoustical parameter for the specific portion of a building.

The airborne and/or aerosol pathogens risk exposure may then be determined as a function of the recorded sound pressure level over time and the determined acoustical parameter. At a same recorded sound pressure level different risks may be found depending on the determined acoustical parameter. According to one example, the recorded sound pressure level is at a level of 61 dB. In a portion of the building having a determined sound strength parameter, G, of 25 (an indication of a reverberant room) the risk is determined to be relatively low. However, in a portion of a building having a determined sound strength parameter, G, of 15 (an indication of a damped room) the risk is determined to be relatively high.

It is noted that the acoustical parameter, e.g. G, for the portion 102 of the building may be determined, e.g. using one or more microphones of one or more mobile electronic devices present in the portion 102 of the building upon it is decided that a determination of an airborne and/or aerosol pathogen risk exposure is to be performed. Alternatively, or in combination, an acoustical parameter, e.g. G, for the portion 102 of the building stored in the server 106 may be used for determining the airborne and/or aerosol pathogen risk exposure.

The function used for determining the airborne and/or aerosol pathogens risk exposure may be referred to as a risk evaluation function. The risk evaluation function may be a correlation function computed with some regression model. It may also be algorithm providing the corresponding value of probability level from a given sound pressure level from a dedicated database. Alternatively, or in combination, various supervised machine learning based regression analysis may be used to compute the function. For example, it may be a Ridge regression, a Random Forest regression, a decision tree regression, a gradient boosting regression, a support vector machine regression, a Lasso regression or a neural network based regression analysis. Many programming and software resources or modules are available in the prior art. For example, if all or part of the function is computer implement in Python programming language, Scikit-learn, Keras or TensorFlow modules provide useful and ready to use API for machine learning algorithms.

The risk evaluation function may be executed on an electronic mobile device 204. Alternatively, or in combination, the risk evaluation function may be executed on the server 106. Part of the risk evaluation function may be executed on an electronic mobile device 204 and part of the risk evaluation function may be executed on the server 106.

The risk evaluation function may be executed by data processing circuitry. The circuitry may be comprised in one or more electronic mobile devices 204 and/or the server 106. The circuitry may comprise one or more processors. Such one or more processors can be instructed to carry out sequences of arithmetic or logical operations to perform tasks or actions. The circuitry may further comprise dedicated hardware portion(s) to carry out sequences of arithmetic or logical operations to perform tasks or actions. The circuitry may further comprise a controller configured to perform control the one or more processors and/or dedicated hardware portion(s). The circuitry may further comprise other electronic components like input/output interfaces, non-volatile or volatile digital storages media, and buses that are communication systems for the data transfer between components inside the circuitry. One of the input/output devices may be user interface for human-machine interaction, for example graphical user interface to display human understandable information. One of the input/output devices may be a microphone located in a mobile electronic device.

Further, a muffled or unmuffled condition of the microphone of a mobile electronic device 204 may be determined. This may e.g. be made using signal analysis of a signal recorded by the microphone. Such signal analysis may e.g. ne configured to recognizes the associated users voice. Combined with historic data the it may be detected, at any moment, if the mobile electronic device 204 is exposed (unmuffled condition) or in a pocket/hand bag, etc. (muffled condition). The risk evaluation function may also depend on the muffled/unmuffled condition as a factor. Hence, a muffled condition may be compensated for when executing the risk evaluation function. Alternatively, upon the mobile electronic device 204 is in the muffled condition it may not be used for recording sound pressure level(s).

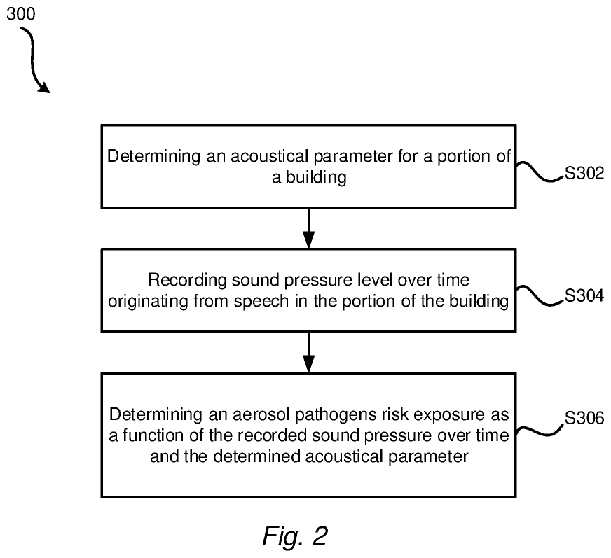

With reference to FIG. 2 a computer implemented method 300 for determining an airborne and/or aerosol pathogen risk exposure will be discussed. The method 300 comprising the following acts. The acts may be performed in any suitable order.

Determining S302 an acoustical parameter for a portion 102 of a building. Various methods for determining S302 an acoustical parameter for the portion 102 of the building are discussed above. In order to avoid undue repetition, reference is made to the above.

Recording S304 sound pressure level over time originating from speech in the portion of the building. The recording S304 is preferably made by means of a microphone in a mobile electronic device 204 located in the portion 102 of the building. The recording S304 of sound pressure level are discussed above. In order to avoid undue repetition, reference is made to the above.

Determining S306 the airborne and/or aerosol pathogens risk exposure as a function of the recorded sound pressure over time and the determined acoustical parameter. The determining S306 of sound pressure level are discussed above. In order to avoid undue repetition, reference is made to the above.

The method may further comprise determining a muffled or unmuffled condition of the microphone. Upon determining a muffled condition, the act of determining the airborne and/or aerosol pathogens risk exposure may comprise compensating for the muffled condition.

The method may further comprise logging time resolved presence information for persons being present in the portion of the building by identifying presence of mobile electronic devices, associated with the respective person, in the portion of the building.

The method may further comprise registering whether a person being present in the portion of the building was infected by a disease while being in the portion of the building. Thus, if it is determined that a person who was present in the portion of the building at that time had been infected by the disease, this is information is registered. Upon a person being present in the portion of the building was infected by a disease, the act of determining the airborne and/or aerosol pathogens risk exposure as a function of the logged sound pressure over time and the determined acoustical parameter may be performed.

The person skilled in the art realizes that the present invention by no means is limited to the preferred embodiments described above. On the contrary, many modifications and variations are possible within the scope of the appended claims.

For example, in an indoor environment, the sound pressure level of human voice during speech depends on many factors, such as the size of the indoor room, the amount of sound absorption in the room, and the number of persons in the room. Hence a size of the portion of the building and/or a number of persons present in the portion of the building may be used as factors in the risk evaluation function. The size and/or the number of persons may be used as biasing factors in the risk evaluation function.

Additionally, variations to the disclosed embodiments can be understood and effected by the skilled person in practicing the claimed invention, from a study of the drawings, the disclosure, and the appended claims.

1-14. (canceled)

15. A computer implemented method for determining an airborne and/or aerosol pathogen risk exposure, the method comprising:
logging time resolved presence information for persons being present in a portion of a building by identifying presence of mobile electronic devices associated with a respective one of the persons in the portion of the building, wherein presence of at least one of the mobile electronic devices in the portion of the building is determined using a wireless communication-based positioning function;
determining an acoustical parameter related to acoustical damping present in the portion of the building;
recording, using a microphone in at least one of the mobile electronic devices located in the portion of the building, individual sound pressure levels over time originating from speech from different persons being present in the portion of the building;
registering whether a person being present in the portion of the building was infected by a disease while being in the portion of the building; and
upon a person being infected by the disease while present in the portion of the building, determining the airborne and/or aerosol pathogens risk exposure as a function of the recorded sound pressure levels over time and the determined acoustical parameter.

16. The method according to claim 15, wherein determining the acoustical parameter in the portion of the building is performed using the microphone of the at least one of the mobile electronic devices located in the portion of the building.

17. The method according to claim 15, further comprising determining a muffled or unmuffled condition of each microphone, wherein, upon determining a muffled condition, determining the airborne and/or aerosol pathogens risk exposure comprises compensating for the muffled condition.

18. The method according to claim 15, wherein recording is performed using a plurality of microphones in a plurality of the mobile electronic devices located in the portion of the building.

19. The method according to claim 15, wherein the at least one of the mobile electronic devices comprises one or more of: a mobile phone, a smart watch, a laptop, and a tablet.

20. A non-transitory computer readable medium storing instructions which when executed by a processor cause the processer to perform the method according to claim 15.

21. An electronic system configured to determine an airborne and/or aerosol pathogen risk exposure, the electronic system comprising:
a microphone; and
circuitry configured to execute:
a presence logging function configured to log time resolved presence information for persons being present in a portion of a building by identifying presence of mobile electronic devices associated with a respective one of the persons in the portion of the building, wherein presence of at least one of the mobile electronic devices in the portion of the building is determined using a wireless communication-based positioning function;
an acoustical parameter determining function configured to determine an acoustical parameter related to acoustical damping present in the portion of the building;
a sound recording function configured to record, using the microphone, individual sound pressure levels over time originating from speech from different persons being present in the portion of the building;
an infection registering function configured to register whether a person being present in the portion of the building was infected by a disease while being in the portion of the building; and
a risk evaluation function configured to, upon a person being infected by the disease while present in the portion of the building, determine the airborne and/or aerosol pathogens risk exposure as a function of the recorded individual sound pressure levels over time and the determined acoustical parameter.

22. The electronic system according to claim 21, wherein the microphone is comprised in the at least one of the mobile electronic devices located in the portion of the building.

23. The electronic system according to claim 22, wherein the circuitry is comprised in the at least one of the mobile electronic devices.

24. The electronic system according to claim 22, wherein the circuitry is comprised in a server being in wireless communication with the at least one of the mobile electronic devices.

25. The electronic system according to claim 22, wherein the circuitry is in part comprised in the at least one of the mobile electronic devices and in part comprised in a server being in wireless communication with the at least one of the mobile electronic devices.
